(19) United States
(12) Patent Application Publication
(10) Pub. No.: US 2023/0183629 A1
(43) Pub. Date: Jun. 15, 2023

(54) BIOREACTOR AND METHOD FOR CULTIVATING CELLS BY USING THE SAME

(71) Applicant: Pensees Inc., Seoul (KR)

(72) Inventor: Sung Jun Lee, Gimhae-Si (KR)

(73) Assignee: Pensees Inc., Seoul (KR)

(21) Appl. No.: 17/646,165

(22) Filed: Dec. 28, 2021

(30) Foreign Application Priority Data
Dec. 10, 2021 (KR) 10-2021-0177008

(51) Int. Cl. C12M 3/04; C12M 3/00; C12M 1/34; C12M 1/00

(52) U.S. Cl. CPC C12M 27/12; C12M 23/48; C12M 41/42; C12M 23/50

(57) ABSTRACT

Disclosed is a bioreactor including a rotation device that rotates a reaction container while a reference direction is taken as an axial direction thereof, and an angle adjusting device coupled to the rotation device to adjust an angle defined by the reference direction, which is dependent on an arrangement posture of the rotation device, with respect to a ground surface, by changing the arrangement posture of the rotation device.

### CROSS-REFERENCE TO RELATED APPLICATION

This application claims the benefit of priority to Korean Patent Application No. 10-2021-0177008, filed in the Korean Intellectual Property Office on Dec. 10, 2021, the entire contents of which are incorporated herein by reference.

### TECHNICAL FIELD

The present disclosure relates to a bioreactor, and more particularly, to a bioreactor for cultivating cells and a method for cultivating cells by using the same.

### BACKGROUND

The bioreactor refers to a machine device that is manufactured and designed in an engineering way to create a biological activation environment. The bioreactor is like a container that generates a chemical reaction of organic materials extracted from a living body or materials activated biochemically. The process may be aerobic or anaerobic. With reference to operation schemes, bioreactors are classified into a batch type, a fed-batch type, and a continuous type (e.g., a continuous agitation type reactor and the like). An example of a continuous cultivator includes a chemostat.

The bioreactor may be used to cultivate cells. When media and cells are introduced into the bioreactor, the bioreactor agitates the media containing cells to induce growth of the cells through exchange of materials.

A scheme that may be used for the bioreactor for cultivating the cells includes an impeller type. The impeller type is a type of generating flows of a fluid in an interior of a reaction vessel by rotating an impeller having wings disposed in the interior of the reaction vessel. An apparatus for a large-scale reaction may be designed when the impeller type is used, but cells are damaged by the impeller as the impeller directly pushes the fluid such that the fluid flows, and the entire reaction vessel has to be washed and sterilized at every batch thereof to be used again.

The bioreactor for cultivating cells may include an orbital shaker scheme. The orbital shaker scheme is a type of rotating a reaction vessel along an orbit in conjunction with rotation of a support body by rotating the support body, in which the reaction vessel is seated. In this case, it is easy to replace the reaction vessel for the use of the reaction vessel at every batch thereof, but it requires a very high RPM to cause the cells to be damaged in the process, and it is difficult to use the reaction vessel of a large scale.

### SUMMARY

The present disclosure has been made to solve the above-mentioned problems occurring in the prior art while advantages achieved by the prior art are maintained intact.

An aspect of the present disclosure provides a bioreactor that prevents sediments and causes cells to grow up easily without being damaged, and a method for cultivating cells by using the same.

The technical problems to be solved by the present disclosure are not limited to the aforementioned problems, and any other technical problems not mentioned herein will be clearly understood from the following description by those skilled in the art to which the present disclosure pertains.

According to an aspect of the present disclosure, a bioreactor includes a rotation device that rotates a reaction container while a reference direction is taken as an axial direction thereof, and an angle adjusting device coupled to the rotation device to adjust an angle defined by the reference direction, which is dependent on an arrangement posture of the rotation device, with respect to a ground surface, by changing the arrangement posture of the rotation device.

According to another aspect of the present disclosure, a method for cultivating cells includes arranging a reaction container, in which cells and a medium are embedded, along a reference direction that is one direction that crosses an upward/downward direction, rotating the reaction container along a first direction that is one direction, in which the reaction container is rotated while the reference direction is taken as an axial direction thereof, stopping rotating the reaction container, rotating the reaction container along a second direction that is an opposite direction to the first direction, and stopping rotating the reaction container.

According to another aspect of the present disclosure, a method for cultivating cells includes arranging a reaction container, in which cells and a medium are embedded, along a reference direction that is one direction that is inclined with respect to a ground surface, rotating the reaction container while the reference direction is taken as an axial direction thereof, and inclining the reaction container such that an angle defined by the reference direction with respect to the ground surface decreases at a specific cycle.

### BRIEF DESCRIPTION OF THE DRAWINGS

The above and other objects, features and advantages of the present disclosure will be more apparent from the following detailed description taken in conjunction with the accompanying drawings:

FIG. 1 is a view conceptually illustrating a reaction container used for a bioreactor and materials accommodated in the reaction container according to an embodiment of the present disclosure;

FIG. 2 is a longitudinal sectional view of a reaction container used for a bioreactor according to an embodiment of the present disclosure;

FIG. 3 is a perspective view illustrating a state, in which a rotation device of a bioreactor is not completely laid, according to an embodiment of the present disclosure;

FIG. 4 is a view illustrating a right side surface of a bioreactor of FIG. 3;

FIG. 5 is a plan view of a bioreactor of FIG. 3;

FIG. 6 is a view illustrating a rear surface of a bioreactor of FIG. 3;

FIG. 7 is a view illustrating a front surface of a bioreactor of FIG. 3;

FIG. 8 is a perspective view illustrating a state, in which a rotation device of a bioreactor is completely laid, according to an embodiment of the present disclosure; and

FIG. 9 is a view illustrating a right side surface of a bioreactor of FIG. 8.

### DETAILED DESCRIPTION

Hereinafter, some embodiments of the present disclosure will be described in detail with reference to the exemplary drawings. Throughout the specification, it is noted that the same or like reference numerals denote the same or like components even though they are provided in different drawings. Further, in the following description of the present disclosure, a detailed description of known functions and configurations incorporated herein will be omitted when it may make the subject matter of the present disclosure rather unclear.

In addition, terms, such as first, second, A, B, (a), (b) or the like may be used herein when describing components of the present disclosure. The terms are provided only to distinguish the components from other components, and the essences, sequences, orders, and the like of the components are not limited by the terms. When it is described that one element is connected, coupled, or electrically connected to another element, the element may be directly connected or coupled to the other element, but a third element may be connected, coupled, or electrically connected between the elements.

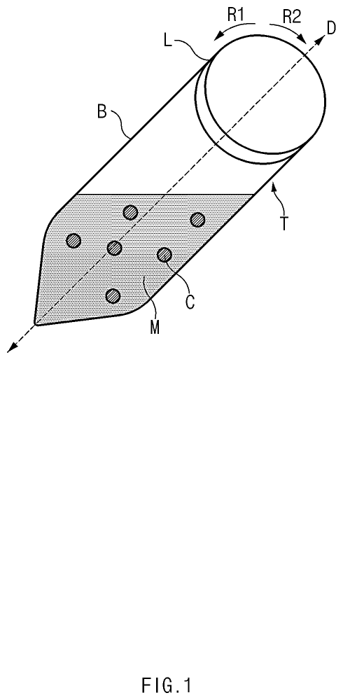

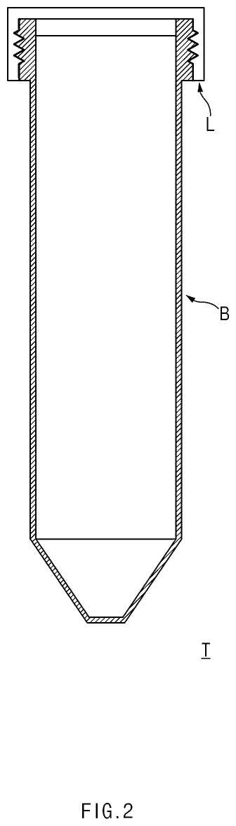

FIG. 1 is a view conceptually illustrating a reaction container “T” used for a bioreactor 1 and materials accommodated in the reaction container “T” according to an embodiment of the present disclosure. FIG. 2 is a longitudinal sectional view of the reaction container “T” used for the bioreactor 1 according to an embodiment of the present disclosure.

The reaction container “T” may be a conical tube. The reaction container “T” may include a container body “B” and a lid “L”. The container body “B” may include a cylindrical part having an opening at an upper end thereof, and a conical part connected to a lower end of the cylindrical part. The lid “L” may be coupled to an upper end of the container body “B” to be detachable to open and close the opening of the container body “B”. A screw thread may be formed in an interior of the lid “L”, and a screw thread corresponding to the screw thread in the interior of the lid “L” may be foamed at an upper end of the container body “B” such that the container body “B” and the lid “L” are firmly screw-coupled to each other. Materials may be introduced into the container body “B” or discharged to an outside through the opening of the container body “B”. Mediums “M” and Cells “C” for cultivation may be accommodated in the reaction container “T”.

A reference direction “D” may be a direction that is parallel to a lengthwise direction of the reaction container “T”. The reaction container “T” may be rotated by a rotation device 10, which will be described below, while the reference direction “D” is taken as an axial direction thereof. Let's assume that one of directions, in which the reaction container “T” is rotated while the reference direction “D” is taken as the axial direction thereof, is a first direction R1 and an opposite direction to the first direction R1 is a second direction R2. The bioreactor 1 according to the embodiment of the present disclosure may rotate the reaction container “T” in at least one of the first direction R1 and the second direction R2.

An agitator (not illustrated) for agitating contents may be provided in an interior of the reaction container “T”. The agitator may not be coupled to an inner surface of the reaction container “T” and may freely float on the contents. The agitator may be freely rotated due to the inertia thereof to agitate the contents better when the reaction container “T” is rotated. The agitator may have a bar shape, and may have a “t” shape.

Although not illustrated in the drawings of the specification of the present disclosure, a hole may be formed in an air circulation lid that is a modification of the lid “L”. A plurality of holes may be formed. The hole may be formed in the air circulation lid such that oxygen that is necessary for growth of the cells “C” may be consistently supplied. A filter membrane may be disposed in the hole to prevent penetration of germs and viruses. When the lid “L” is loosely coupled to the container body “B” so that a sealing state is not maintained even when a hole is not formed in the lid “L”, oxygen may be supplied as if there were a hole.

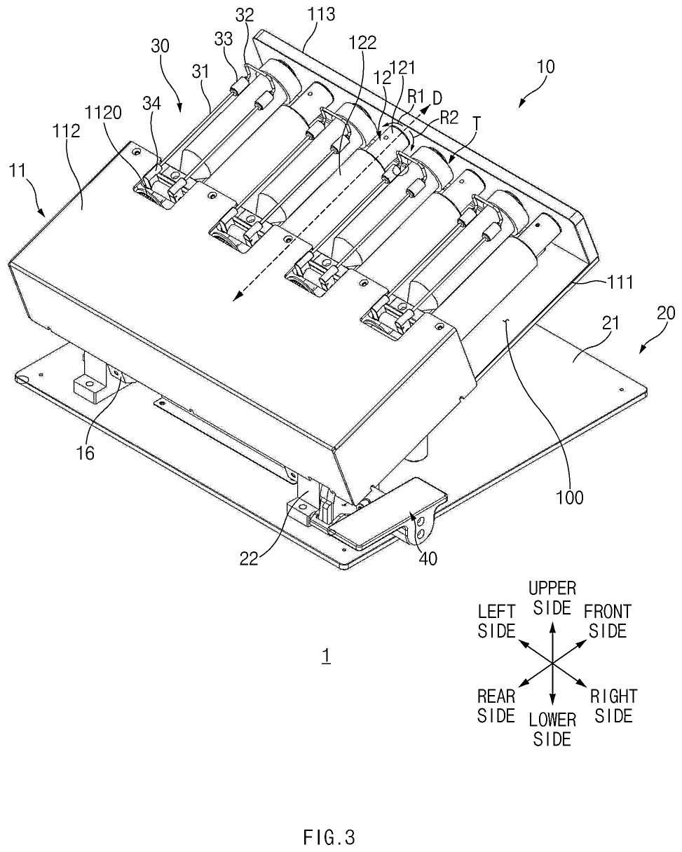

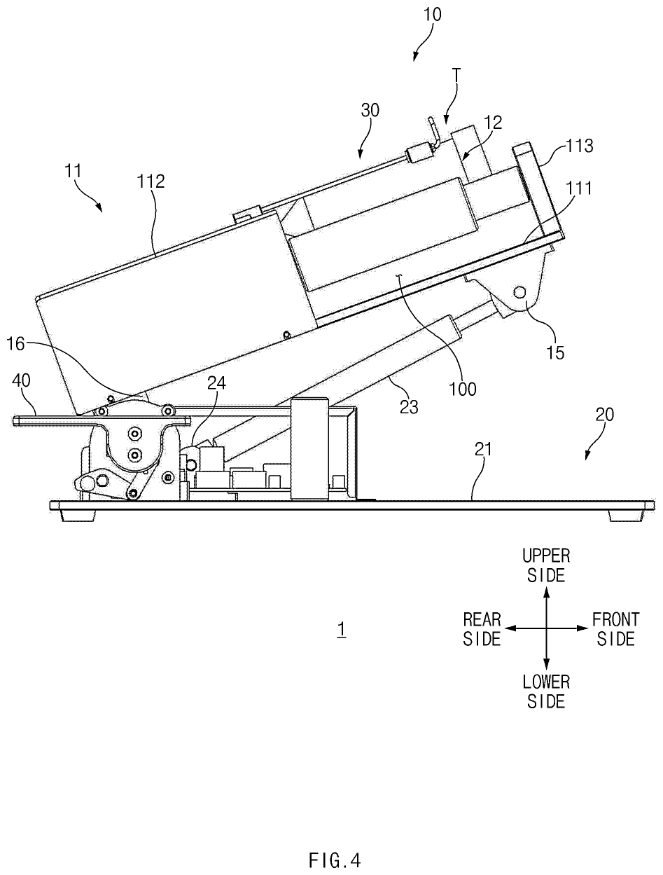

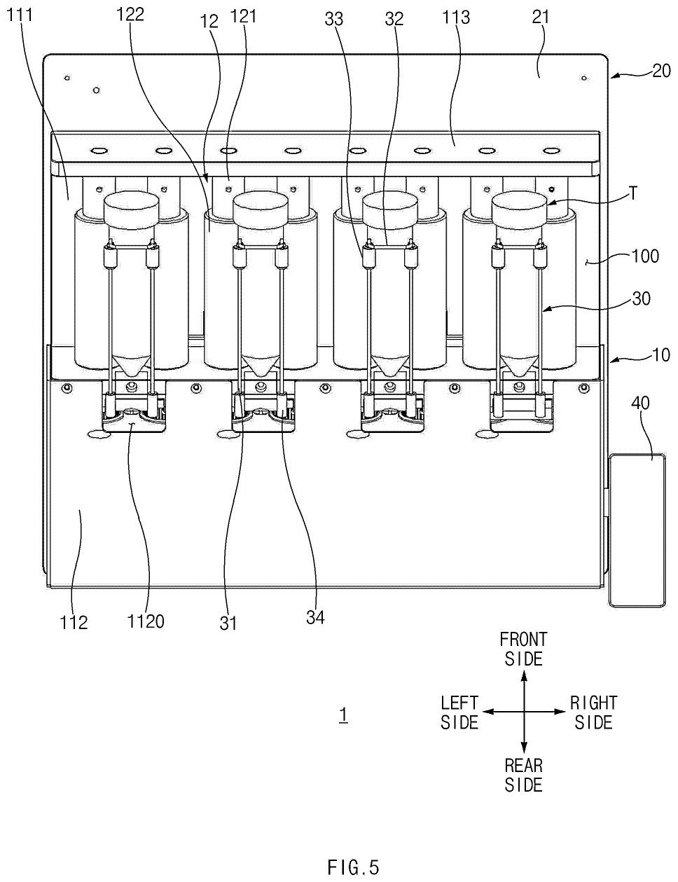

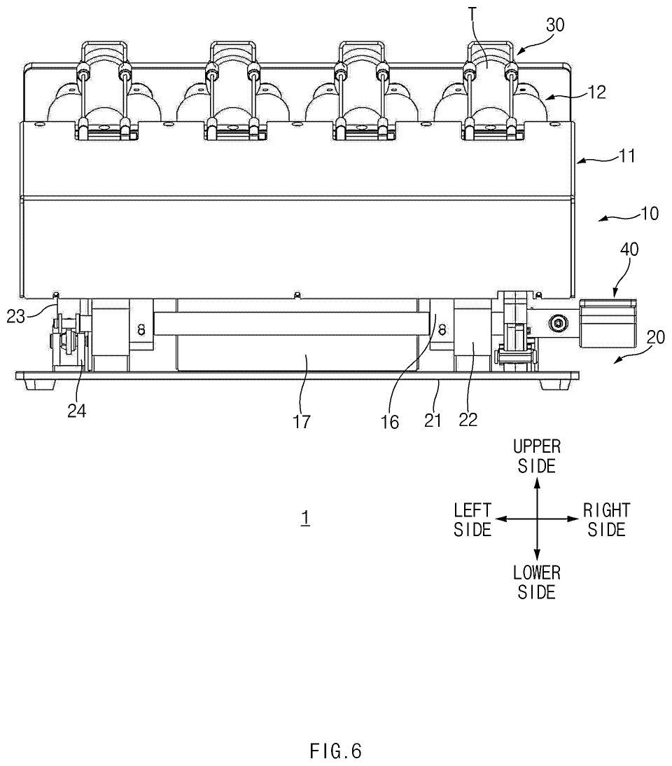

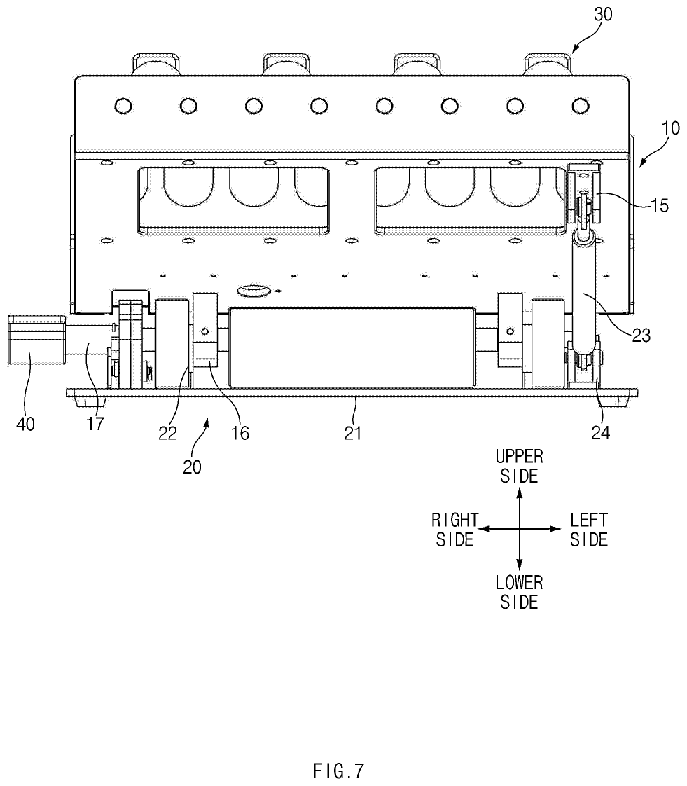

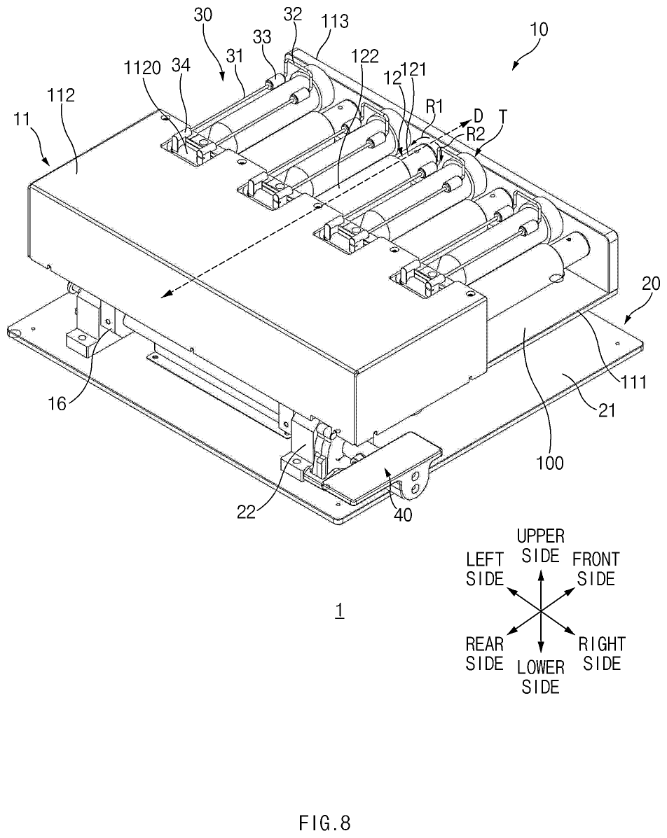

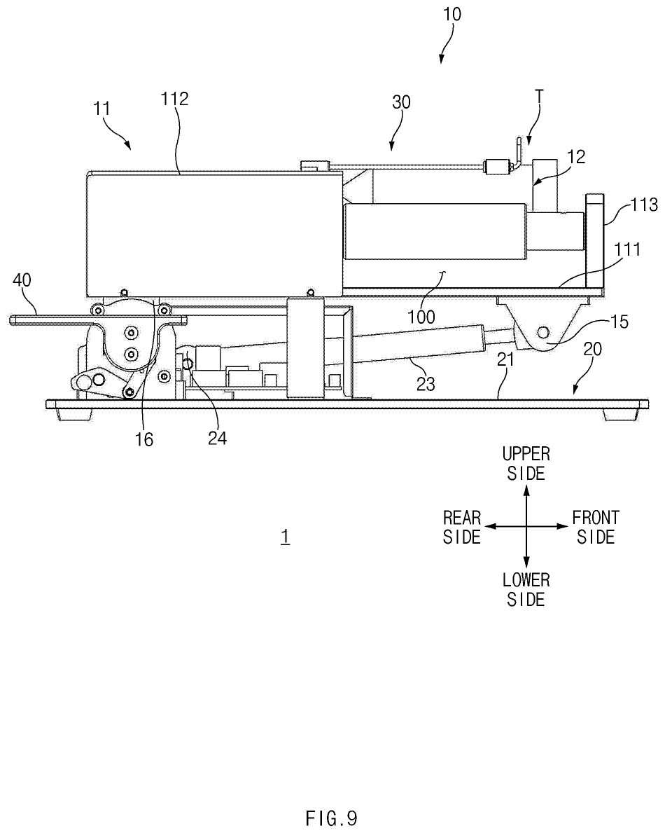

FIG. 3 is a perspective view illustrating a state, in which the rotation device 10 of the bioreactor 1 is not completely laid, according to an embodiment of the present disclosure. FIG. 4 is a view illustrating a right side surface of the bioreactor 1 of FIG. 3. FIG. 5 is a plan view of the bioreactor 1 of FIG. 3. FIG. 6 is a view illustrating a rear surface of the bioreactor 1 of FIG. 3. FIG. 7 is a view illustrating a front surface of the bioreactor 1 of FIG. 3. FIG. 8 is a perspective view illustrating a state, in which the rotation device 10 of the bioreactor 1 is completely laid, according to an embodiment of the present disclosure. FIG. 9 is a view illustrating a right side surface of the bioreactor 1 of FIG. 8.

Referring to the drawings, the bioreactor 1 according to the embodiment of the present disclosure may include the rotation device 10 and an angle adjusting device 20. In the specification and the drawings of the present disclosure, a leftward/rightward direction and a forward/rearward direction are directions that are perpendicular each other and to an upward/downward direction and are relative directions used for convenience of description, and may become different according to a posture of the bioreactor 1.

Rotation Device 10

The rotation device 10 is an element that rotates the reaction container “T” while the reference direction “D” is taken as an axial direction. The reaction container “T” may be fixed to the rotation device 10, and the reaction container “T” may be rotated along the first direction R1 or the second direction R2 by the rotation device 10. The reaction container “T” may be fixed to the rotation device 10 such that the conical part faces a rear side.

The rotation device 10 may include a rotation frame 11 and a plurality of tube rollers 12. The tube roller 12 may rotate the reaction container “T” that contacts an upper side thereof by using frictional forces. The reaction container “T” may be disposed between adjacent tube rollers 12, and the reaction container “T” may receive frictional forces to be rotated by the adjacent tube rollers 12 that are rotated in opposite directions.

The tube rollers 12 may be rotatably coupled to the rotation frame 11 to be rotated while the reference direction “D” is taken as an axial direction thereof. Accordingly, the plurality of tube rollers 12 may extend along the reference direction “D”, and may be arranged along a direction that is perpendicular to the reference direction “D”. As may be seen in the drawings, the plurality of tube rollers 12 may be spaced apart from each other along the leftward/rightward direction.

The rotation frame 11 may be recessed to form a frame recess 100. The plurality of tube rollers 12 may be disposed in the frame recess 100. The rotation frame 11 may include a plate-shaped frame base plate 111, a frame front cover 113 and a frame rear cover 112 coupled to an upper surface of the frame base plate 111. Elements, such as a motor and a processor, for driving the rotation device 10 may be accommodated between the frame rear cover 112 coupled to a rear portion of the frame base plate 111 and the frame base plate 111. The frame front cover 113 coupled to a front portion of the frame base plate 111 may be spaced apart from each other from the frame rear cover 112 along the forward/rearward direction. A space between the frame front cover 113 and the frame rear cover 112 may be the frame recess 100. A front end of the tube roller 12 may be rotatably coupled to the frame front cover 113, and a rear end of the tube roller 12 may be rotatably coupled to the frame rear cover 112.

The front end of the frame rear cover 112 may be recessed rearwards, and a frame rear recess 1120 may be formed in the frame rear cover 112. The frame rear recess 1120 is a part that is formed by removing a portion of the frame rear cover 112 such that rotation of a clamp part 30, which will be described below, is not hampered when the clamp part 30 is rotated. The frame rear recess 1120 may be connected to the frame recess 100.

The tube roller 12 may include a roller axial member 121 and a roller cover 122. The roller cover 122 may surround the cylindrical roller axial member 121. When the roller axial member 121 is rotated, the roller cover 122 may be rotated together to rotate the reaction container “T” in contact by using frictional forces. Because a step is formed by a difference of diameters of the roller axial member 121 and the roller cover 122, the roller cover 122 may contact the container body “B” as a whole in a state, in which the lid “L” of the reaction container “T” does not contact the roller cover 122. This is because the lid “L” of the reaction container “T” is located on a front side of the tube roller 12, on which only the roller axial member 121 is disposed and the roller cover 122 is not formed. When the container body “B” and the lid “L” are rotated while contacting the tube rollers 12 having the same diameter as a whole, the container body “B” fails to contact the roller cover 122 as a whole and a difference is caused between rotational speeds of the container body “B” and the lid “L” so that the lid “L” may be opened.

Elements connected to the angle adjusting device 20 for rotation of the rotation device 10 may be disposed on a lower surface of the frame base plate 111. A driving connector 15 may protrude downwards from a front side of a lower surface of the frame base plate 111. The driving connector 15 may be rotatably coupled to a rotation driving member 23, which will be described below.

A rotation connector 16 may protrude downwards from a rear side of the lower surface of the frame base plate 111. Two rotation connectors 16 may be provided to be spaced apart from each other along the leftward/rightward direction, and may be connected to each other through a frame axial member 17 that extends along the leftward/rightward direction. The rotation connector 16 and the frame axial member 17 may be rotatably coupled to a hinge 22, which will be described below.

Clamp Part 30

The bioreactor 1 according to an embodiment of the present disclosure may include the clamp part 30. The clamp part 30 may press the reaction container “T” against the plurality of tube rollers 12 to contact the reaction container “T” to the plurality of tube rollers 12. The clamp part 30 may be coupled to the rotation device 10 to perform a pressing operation.

The clamp part 30 may be rotatably coupled to the rotation device 10. Accordingly, the clamp part 30 may selectively have a fixed state, in which the reaction container “T” is pressed against the plurality of tube rollers 12, and a separation state, in which the plurality of tube rollers 12 are separated from the reaction container “T”. A user may manipulate the clamp part 30 to selectively press the reaction container “T” against the tube rollers 12 to prevent the reaction container “T” from being separated from the rotation device 10 or separate the reaction container “T” from the rotation device 10.

The clamp part 30 may include a clamp frame 31 that extends along the reference direction “D”. A plurality of clamp frames 31 may be provided, and may be spaced apart from each other along the leftward/rightward direction. Accordingly, the clamp part 30 may press the reaction container “T” at a specific number of sites to fix the reaction container “T” without covering an entire surface of the reaction container “T”.

The clamp part 30 may include a clamp connector 34. The clamp connector 34 is connected to one end of the clamp frame 31 in the reference direction “D”, and is rotatably coupled to the rotation device 10. One end of the clamp frame 31 in the reference direction “D” may be a rear end of the clamp frame 31. The clamp connector 34 may be inserted into the frame rear recess 1120. The clamp connector 34 may be rotatably coupled to the rotation device 10 while the leftward/rightward direction is taken as an axial direction thereof. Accordingly, as the clamp part 30 is rotated about the clamp connector 34 because it is rotatably coupled to the rotation device 10 at a portion, at which the clamp connector 34 is located, as in the drawings, a bent part 32 and the clamp frame 31 may be moved from a location, at which the reaction container “T” is pressed to an upper side of the reaction container “T” to be disposed at a location that is spaced apart from the reaction container “T”. The locations are locations of the clamp part 30 in the fixed state and the separation state, which have been described above.

The clamp part 30 may include the bent part 32. The bent part 32 may extend from an opposite end of the clamp frame 31 in the reference direction “D” along a direction that crosses the reference direction “D”. With reference to the state of FIG. 8, in which the rotation device 10 is completely laid, the bent part 32 may extend upwards from a front end, that is, an opposite end or the clamp frame 31 in the reference direction “D”. The bent part 32 may connect the plurality of clamp frames 31. The user may easily grip the bent part 32 to easily manipulate the clamp part 30.

The clamp part 30 may include a clamp roller 33. The clamp roller 33 may be rotatably coupled to the clamp frame 31 to support the reaction container “T”. The clamp roller 33 may contact an upper side of the reaction container “T” to be rotated about the clamp frame 31 by using frictional forces when the reaction container “T” is rotated. A plurality of clamp rollers 33 may be provided, and may be disposed in the plurality of clamp frames 31.

Angle Adjusting Device 20

The angle adjusting device 20 is coupled to the rotation device 10 such that an angle defined by the reference direction “D” with respect to a ground surface may be adjusted, by changing an arrangement posture of the rotation device 10. The reference direction “D” is a direction that is dependent on the arrangement posture of the rotation device 10. Accordingly, in FIGS. 3 to 7, a direction that is inclined upwards with respect to the front side and an opposite direction thereto become the reference direction “D”, and in FIGS. 8 and 9, in which the arrangement posture of the rotation device 10 is changed, the forward/rearward direction becomes the reference direction “D”.

The angle defined by the reference direction “D” with respect to the ground surface may be reduced by using the angle adjusting device 20 as sizes of the cell “C” lumps become lager, whereby sediments may be effectively prevented. During the rotation, the cell “C” lumps continue to grow up and the sizes of thereof become larger. As the sizes of the cell “C” lumps become larger, the sediments occur well, and when the angle of the rotation device 10 with respect to the ground surface is reduced, the tendency of the sediments may be alleviated without increasing a rotational speed of the reaction container “T”. That is, sediment of the cell “C” lumps may not occur when a value obtained by adding, among drags generated in the cell “C” lumps as the rotation device 10 rotates the reaction container “T”, a drag having a value that is largest on the upper side, and a buoyant force generated in the cell “C” lumps by the medium “M” is equal to or larger than a magnitude of a gravitational force acting on the cell “C” lumps, the rotation device 10 may be inclined by the angle adjusting device 20 according to an angle that is found to prevent sediments.

The angle adjusting device 20 may include an angle adjusting base plate 21 configured to contact the ground surface while being perpendicular to the upward/downward direction. The angle adjusting device 20 may include the frame hinge 22, to which one end of the rotation device 10 in the reference direction “D” is rotatably coupled. The frame hinge 22 may be coupled to an upper surface of the angle adjusting base plate 21. One end of the rotation device 10, which is coupled to the frame hinge 22, is the rotation connector 16, which has been described above. Accordingly, the rotation device 10 may be rotated about an imaginary axis that crosses a location, at which the frame hinge 22 and the rotation connector 16 are coupled to each other, in the leftward/rightward direction, with respect to the angle adjusting device 20. According to the rotation, a location of the frame front cover 113 in the upward/downward direction and the forward/rearward direction, which corresponds to an opposite end of the rotation device 10 in the reference direction “D”, may be changed, and thus the reference direction “D” may be changed.

The angle adjusting device 20 may include the rotation driving member 23. The rotation driving member 23 may be coupled to an opposite end of the rotation device 10 to push or pull the opposite end of the rotation device 10 with respect to the reference direction “D” such that the arrangement posture of the rotation device 10 is changed by rotating the rotation device 10 about the frame hinge 22. Here, the opposite end of the rotation device 10 is the above-described driving connector 15. The rotation driving member 23 may be rotatably coupled to the driving connector 15. The rotation driving member 23 may be coupled to a driving hinge 24 rotatably coupled to an upper surface of the angle adjusting base plate 21.

The rotation driving member 23 may include a hydraulic cylinder that may be expandable and contractible along the extension direction. However, the expandable/contractible device is not limited the hydraulic cylinder. As the rotation driving member 23 is contracted, the rotation device 10 may be pushed out from the angle adjusting base plate 21 or be pulled toward the angle adjusting base plate 21.

The rotation driving member 23, as illustrated, may be disposed to be inclined upwards with respect to the front side. A front end of the rotation driving member 23 may be rotatably coupled to the driving connector 15, and a rear end of the rotation driving member 23 may be rotatably coupled to the driving hinge 24. Accordingly, the rotation driving member 23, the rotation frame 11, and the angle adjusting base plate 21 are rotatably coupled to each other to form a 3-link shape that defines a triangle, and the rotation driving member 23 may be expanded or contracted and the shape of the link shape may be changed.

Handle 40

The bioreactor 1 according to the embodiment of the present disclosure may include a handle 40. The handle 40 is connected to one end of the rotation device 10. Here, the one end of the rotation device 10 is the above-described rotation connector 16. The handle 40 may be disposed on an outside of the rotation device 10 along the leftward/rightward direction. The handle 40 may have a flat upper surface, and may have a part that protrudes rearwards. Accordingly, the user may adjust the angle defined by the rotation device 10 with respect to the angle adjusting device 20 by manipulating the handle 40.

Processor

The processor is configured to control an operation of rotating the reaction container “T” by the rotation device 10. The processor may be installed inside the frame rear cover 112 of the rotation device 10. The processor is a constituent element including an element that may perform logical operations for performing a control command, and may include a central processing unit (CPU). The processor may be connected to the elements to transmit signals for controls according to the control commands to the element, and may be connected to the sensors and the acquirers to receive the acquired information in a form of signals. Accordingly, in the embodiment of the present disclosure, the processor may be electrically connected to various elements included in the bioreactor 1. Because the processor may be electrically connected to the elements, it may be connected to the elements by wire or may further include a communication module that may perform communication wirelessly for mutual communications.

The bioreactor 1 may further include a storage medium, and control commands performed by the processor may be stored in the storage medium to be utilized. The storage medium may be a device such as a hard disk drive (HDD), a solid state drive (SSD), a server, a volatile medium, or a nonvolatile medium, but the kinds thereof are not limited thereto. In addition, the storage medium may further store data that is necessary to allow the processor to perform an operation.

The processor is electrically connected to the rotation device 10. Here, the electrical connection includes physical connection for current flows and connection through communication for transmitting and receiving control signals. The processor controls rotating or stopping the reaction container “T” by the tube rollers 12 by controlling the rotation of the tube rollers 12 by the rotation device 10.

The processor may control the rotation device 10 to rotate the reaction container “T” along the first direction R1, stop an operation of rotating the reaction container “T”, rotating the reaction container “T” along the second direction R2, and stop an operation of rotating the reaction container “T” in sequence. The processor may control the rotation device 10 to repeat the above-described preset operation. The cells “C” may be made to effectively grow up by repeating a process of rotating the reaction container “T” in one direction, stopping the rotation, rotating it in a reverse direction in turn, and stopping the rotation to exchange material more effectively than when the reaction container “T” continues to be rotated in one direction at the same speed.

A method of cultivating cells by using the above-described preset operation is as follows. The reaction container “T”, in which the cells “C” and the medium “M” are embedded, is arranged along the reference direction “D” that is one direction that crosses the upward/downward direction, and is disposed between adjacent tube rollers 12 of the bioreactor 1. The processor controls the tube rollers 12 to rotate the reaction container “T” along the first direction R1 that is one direction, in which the reference direction “D” is taken as an axial direction thereof. The processor stops the rotation of the reaction container “T”. The processor rotates the reaction container “T” along the second direction R2 that is the opposite direction to the first direction R1. The processor stops the rotation of the reaction container “T”.

The processor may perform an intermittent rotation operation of rotating the reaction container “T” along one of the rotation directions, in which the reference direction “D” is the axial direction. When an intermittent rotation operation is performed, the processor may undergo a process of controlling the rotation device 10 to accelerate a rotational speed of the reaction container “T” to a specific speed, rotate the reaction container “T” according to a specific speed, and decelerate the rotational speed of the reaction container “T” until the reaction container “T” is stopped from the specific speed. When the processor performs the intermittent rotation operation, a time period that is necessary for accelerating the rotational speed of the reaction container “T”, and a time period that is necessary for decelerating the reaction container “T” may be the same. That is, absolute values of the acceleration value during acceleration and the acceleration value during the deceleration may be the same.

The processor may be electrically connected to the angle adjusting device 20. Then angle adjusting device 20 may include a motor, an actuator, a linear actuator, and the like that are electrically controlled to generate a driving force or be deformed to adjust the angle defined by the rotation device 10 with respect to the ground surface. Here, the ground surface means a surface that is perpendicular to the upward/downward direction, and on which the bioreactor 1 is seated. In a state, in which the rotation device 10 is arranged along one direction that is inclined with respect to the ground surface, the reaction container “T” may be fixed to the rotation device 10. Accordingly, the reaction container “T” may be arranged along the reference direction “D” that is inclined with respect to the ground surface. Thereafter, the rotation device 10 rotates the reaction container “T” while the reference direction “D” is taken as an axial direction thereof whereby the cell “C” lumps may grow up. Because a possibility of generating sediments becomes higher as the cell “C” lumps grow up, the processor may control the angle adjusting device 20 to incline the reaction container “T” by changing a posture of the rotation device 10 such that the angle of the reference direction with respect to the ground surface decreases at a specific cycle. Finally, the reaction container “T” may become parallel to the ground surface. A degree, by which the angle decreases at the specific cycle, may be a specific angle that is a fixed value.

In an operation of inclining the reaction container “T”, the angle may not decrease by the specific angle at the cycles, but the angle may decrease according to a proper angle that is found. In detail, at the above-described cycles, the processor may calculate a sediment preventing angle, which is defined by the reference direction “D”, by which a value obtained by adding, among drags generated in the cell “C” lumps due to the rotational speed of the reaction container, a drag having a value that is largest on the upper side, and a buoyant force acting on the cell “C” lumps is made to become equal to a gravitational force acting on the cell “C” lumps, with respect to the ground surface, at the cycle. Data that is a base of the calculation may be fetched from a storage medium by the processor. The processor may fetch data in a table, in which information on volumes and masses of the cell “C” lumps according to duration times of reactions are present, from the storage medium. The processor may incline the reaction container “T” such that the angle of the reference direction with respect to the ground surface decreases to the sediment preventing angle, which is calculated above.

Accordingly, sediments may be prevented and cells may be caused to grow up easily without being damaged.

Although it may have been described until now that all the elements constituting the embodiments of the present disclosure are coupled to one or coupled to be operated, the present disclosure is not essentially limited to the embodiments. That is, without departing from the purpose of the present disclosure, all the elements may be selectively coupled into one or more elements to be operated. Furthermore, because the terms, such as “comprising”, “including”, or “having” may mean that the corresponding element may be included unless there is a specially contradictory description, it should be construed that another element is not extruded but may be further included. In addition, unless defined otherwise, all terms used herein, including technical or scientific terms, have the same meanings as those generally understood by those skilled in the art to which the present disclosure pertains. The terms, such as the terms defined in dictionaries, which are generally used, should be construed to coincide with the context meanings of the related technologies, and are not construed as ideal or excessively formal meanings unless explicitly defined in the present disclosure.

The above description is a simple exemplification of the technical spirits of the present disclosure, and the present disclosure may be variously corrected and modified by those skilled in the art to which the present disclosure pertains without departing from the essential features of the present disclosure. Accordingly, the embodiments disclosed in the present disclosure is not provided to limit the technical spirits of the present disclosure but provided to describe the present disclosure, and the scope of the technical spirits of the present disclosure is not limited by the embodiments. Accordingly, the technical scope of the present disclosure should be construed by the attached claims, and all the technical spirits within the equivalent ranges fall within the scope of the present disclosure.

What is claimed is:

1. A bioreactor comprising:
a rotation device configured to rotate a reaction container while a reference direction is taken as an axial direction thereof; and
an angle adjusting device coupled to the rotation device to adjust an angle defined by the reference direction, which is dependent on an arrangement posture of the rotation device, with respect to a ground surface, by changing the arrangement posture of the rotation device.

2. The bioreactor of claim 1, wherein the rotation device includes:
a plurality of tube rollers rotated while the reference direction is taken as axial directions thereof such that the reaction container in contact with upper sides thereof is rotated by frictional forces.

3. The bioreactor of claim 2, wherein the rotation device further includes:
a rotation frame, to which the plurality of tube rollers are rotatably coupled; and
a frame recess formed on the rotation frame, and
wherein the plurality of tube rollers are disposed in the frame recess.

4. The bioreactor of claim 2, wherein the plurality of tube rollers are spaced apart from each other along a direction that is perpendicular to the reference direction.

5. The bioreactor of claim 2, further comprising:
a clamp part coupled to the rotation device to press the reaction container against a plurality of rollers to contact the reaction container to the plurality of rollers.

6. The bioreactor of claim 5, wherein the clamp part is rotatably coupled to the rotation device to selectively have a fixed state, in which the reaction container is pressed against the plurality of tube rollers, and a separation state, in which the plurality of tube rollers are separated from the reaction container.

7. The bioreactor of claim 6, wherein the clamp part includes:
a clamp frame extending along the reference direction; and
a clamp connector connected to one end of the clamp frame in the reference direction, and rotatably coupled to the rotation device.

8. The bioreactor of claim 7, wherein the clamp part further includes:
a bent part extending from an opposite end of the clamp frame in the reference direction along a direction that crosses the reference direction.

9. The bioreactor of claim 7, wherein the clamp part further includes:
a clamp roller rotatably coupled to the clamp frame so as to support the reaction container.

10. The bioreactor of claim 1, wherein the angle adjusting device includes:
a frame hinge, to which one end of the rotation device is rotatably coupled about the reference direction.

11. The bioreactor of claim 10, wherein the angle adjusting device further includes:
a rotation driving member coupled to an opposite end of the rotation device to push or pull the opposite end of the rotation device with respect to the reference direction whereby the arrangement posture of the rotation device is changed by rotating the rotation device about the frame hinge.

12. The bioreactor of claim 11, wherein the rotation driving member includes:
an expandable/contractible hydraulic cylinder.

13. The bioreactor of claim 10, further comprising:
a handle connected to one end of the rotation device.

14. The bioreactor of claim 1, further comprising:
a processor electrically connected to the rotation device, and
wherein the processor is configured to:
control the rotation device to rotate the reaction container.

15. The bioreactor of claim 14, wherein when it is assumed that one of directions, in which the reaction container is rotated by the rotation device while the reference direction is taken as the axial direction thereof, is a first direction, and an opposite direction thereto is a second direction,
the processor is configured to:
control the rotation device to perform a preset operation of rotating the reaction container along the first direction, stopping rotating the reaction container, rotating the reaction container along the second direction, and stopping rotating the reaction container in sequence.

16. The bioreactor of claim 15, wherein the processor is configured to:
control the rotation device to repeat the preset operation.

17. The bioreactor of claim 14, wherein the processor is configured to:
when an intermittent rotation operation of rotating the reaction container while the reference direction is taken as an axial direction thereof is performed,
control the rotation device to accelerate a rotational speed of the reaction container to a specific speed, rotate the reaction container according to the specific speed, and decelerate the rotational speed of the reaction container until the reaction container is stopped from the specific speed,; and
wherein when the processor performs the intermittent rotation operation, a time period that is necessary for accelerating the rotational speed of the reaction container, and a time period that is necessary for decelerating the reaction container are the same.

18. A method for cultivating cells, the method comprising:
arranging a reaction container, in which cells and a medium are embedded, along a reference direction that is one direction that crosses an upward/downward direction;
rotating the reaction container along a first direction that is one direction, in which the reaction container is rotated while the reference direction is taken as an axial direction thereof;
stopping rotating the reaction container;
rotating the reaction container along a second direction that is an opposite direction to the first direction; and
stopping rotating the reaction container.

19. A method for cultivating cells, the method comprising:
arranging a reaction container, in which cells and a medium are embedded, along a reference direction that is one direction that is inclined with respect to a ground surface;
rotating the reaction container while the reference direction is taken as an axial direction thereof; and
inclining the reaction container such that an angle defined by the reference direction with respect to the ground surface decreases at a specific cycle.

20. The method of claim 19, wherein the inclining of the reaction container includes:
calculating a sediment preventing angle, which is defined by the reference direction, by which a value obtained by adding, among drags generated in the cell lumps due to a rotational speed of the reaction container, a drag having a value that is largest on an upper side, and a buoyant force acting on cells is made to become equal to a gravitational force acting on the cells, with respect to the ground surface, at the cycle; and
inclining the reaction container such that the angle defined by the reference direction with respect to the ground surface decreases to the calculated sediment preventing angle.
